(19) United States
(12) Patent Application Publication
(10) Pub. No.: US 2010/0143313 A1
(43) Pub. Date: Jun. 10, 2010

(54) HOMOGENEOUS DIFFERENTIATION OF HEPATOCYTE-LIKE CELLS FROM EMBRYONIC STEM CELLS

(75) Inventors: Martin L. Yarmush, Newton, MA (US); Cheul H. Cho, Whippany, NJ (US); Arno W. Tilles, Cambridge, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/634,983

(22) Filed: Dec. 10, 2009

Related U.S. Application Data
(60) Provisional application No. 61/121,318, filed on Dec. 10, 2008.

(51) Int. Cl. A61K 35/407; C12N 5/074; A61P 1/16

(52) U.S. Cl. 424/93.7; 435/377

(57) ABSTRACT

One of the major hurdles of cellular therapies for the treatment of liver failure is the low availability of functional human hepatocytes. Although embryonic stem (ES) cells represent a potential cell source for therapy, current methods for differentiation result in mixed cell populations or low yields of the cells of interest. The present invention provides for a rapid, direct differentiation method that yields a homogeneous population of endoderm-like cells with 95% purity. In one embodiment, mouse ES cells cultured on top of collagen-sandwiched hepatocytes differentiate and proliferate into a uniform and homogeneous cell population of endoderm-like cells. The endoderm-like cell population was positive for Foxa2, Sox17 and AFP, and could further differentiate into hepatocyte-like cells that demonstrate hepatic morphology, functionality, and gene and protein expression. Incorporating the hepatocyte-like cells into a bioartificial liver device to treat fulminant hepatic failure improved animal survival, thereby underscoring the therapeutic potential of these cells.

### CROSS REFERENCE TO RELATED APPLICATIONS

This application claims the benefit of priority under 35 U.S.C. §119(e) of U.S. Provisional Application No. 61/121,318 filed Dec. 10, 2008, the content of which is incorporated herein by reference in its entirety.

### FEDERAL FUNDING

This invention was supported by the U.S. government under grant numbers ROI DK43371, K18 DK076819, and K08 DK066040 awarded by the national Institutes of Health. The federal government has certain rights in the invention.

### FIELD OF THE INVENTION

The present invention relates to molecular biology, cell culture, and regenerative medicine. More specifically, this invention describes a novel and reproducible process for differentiating pluripotent stem cells (e.g. embryonic stem cells) into functional hepatocytes.

### BACKGROUND

Liver disease affects millions of people worldwide, and loss of liver function claims 30,000 lives each year in the United States alone. Liver disease manifests itself in many forms such as chronic hepatic, cirrhosis, acute injury, or inherited conditions such as β-1-antitrypsin deficiency. Unlike diseases affecting other endoderm-derived organs such as diabetes (pancreas) or kidney failure which can be treated respectively with insulin injections or dialysis treatments, orthotopic liver transplantation currently remains the only effective mode for treating end-stage liver disease. Approximately 20,000 people in the US are on the waiting list for a liver transplant but with only about 2,500 donor livers available each year, many of these individuals die. Potential alternative approaches may include hepatocyte transplantation, a transplanted tissue engineered liver, or an extracorporeal bio-artificial liver device. The low availability of functional human hepatocytes limits severely these potential therapies, however. Therefore, the generation of a hepatic progenitor cell, with the ability to proliferate in vitro while retaining liver-specific function is a major goal in the field of regenerative medicine.

### SUMMARY OF THE INVENTION

An object of the present invention provides for compositions and methods for rapid, direct differentiation of a homogeneous population of endoderm-like cells.

In one embodiment, embryonic stem cells are cultured on top of collagen-sandwiched hepatocytes such that the stem cells differentiate and proliferate into a uniform and homogeneous cell population of endoderm-like cells that can be further differentiated into hepatocyte-like cells with hepatic morphology, functionality, and gene and protein expression. Another embodiment of the invention provides for hepatocyte-like cells incorporated into a bioartificial liver device. In another embodiment, the bioartificial device containing hepatocyte-like cells is used to treat subjects suffering fulminant hepatic failure.

More specifically, the present invention provides for a rapid, direct differentiation method that yields a homogeneous population of endoderm-like cells with 95% purity. Mouse ES cells were cultured on top of collagen-sandwiched primary rat hepatocytes. Notably, this coculture system directed the differentiation and proliferation of ES cells into a uniform and homogeneous cell population of endoderm-like cells. The morphologically homogeneous cell population expressed transcripts for the major endoderm markers Foxa2, Sox17, and AFP, and could be greatly expanded in culture. The endoderm-like cell population could be further differentiated into hepatocyte-like cells, demonstrating hepatic morphology, functionality, and gene and protein expression. Incorporating the hepatocyte-like cells into a bioartificial liver device to treat fulminant hepatic failure improved animal survival, thereby underscoring the therapeutic potential of these cells.

In one embodiment, ES cell hepatic differentiation was initiated by re-seeding the d10 ES cell-derived endoderm-like (ESDE) cells on collagen gel, followed by a six-day induction period (d14-d19) with 2% dimethyl sulfoxide (DMSO) or 1 μM all-trans retinoic acid (RA) in low glucose (1 g/L) DMEM. DMSO induction resulted in a down-regulation of the endodermal gene Sox17, while an up-regulation of hepatocyte genes tyrosine aminotransferase, glucose-6-phosphatase, and albumin was observed. By day thirty, many of the differentiated ES cells were mono- and bi-nucleated epithelial cells co-expressing cytokeratin-18 and albumin. After thirty days of differentiation with DMSO or RA induction, albumin and urea secretion rates of the ESDE cells were similar to the secretion rates of primary rat hepatocytes. Active glycogen synthesis and drug metabolizing activities were also observed with the ES cell derived hepatocyte-like cells. These hepatocytes are a viable cell source for in vivo transplantation or bioartificial liver devices.

An object of the present invention provides for cells useful in regenerative medicine and cell-based therapies. The invention provides a method for generating a cell source therapy for treating individuals suffering from impaired liver functions. Subsequent to in vitro differentiation, hepatic progenitors or mature hepatocytes may be directly implanted into the individual to restore liver function. An alternative method incorporates the differentiated hepatocytes into an extracorporeal bioartificial liver (BAL) device. Individual cells or hydrogel-encapsulated cells may be seeded into the BAL device. The BAL can be used to sustain an individual with a damaged/diseased liver for long-term therapy or as a short-term therapeutic support prior to liver transplantation.

Another object of the invention provide cells useful for drug screening. Isolated primary hepatocytes, immortalized hepatocytes, and hepatocarcinoma cell lines are routinely used in the pharmaceutical industry to evaluate biological and chemical compounds in drug screening and toxicity assays. The compounds may be evaluated for beneficial or detrimental pharmacological effects on hepatocytes. The effect of the compounds on the phenotype of the differentiated hepatocytes can be assessed by cell viability, morphological changes, alterations in hepatocyte metabolic activity attributable to the applied compound, and/or increased activity of drug detoxifying enzyme cytochrome P450. An example of assessing a beneficial pharmacological effect of a compound on hepatocyte function is the screening of statins (HMG-CoA reductase inhibitors) on hepatocyte clearance of low-density lipoproteins.

Yet another object of the present invention provides for the production of hepatocyte (lipo)proteins. Pluripotent stem (PS) cell-derived differentiated hepatocytes may be used to produce high quantities of hepatocytes proteins for use as diagnostic or therapeutic reagents. Current techniques for the production and/or purification of hepatocyte proteins involve the collection of serum from blood donors or the production of recombinant proteins from transfected cell lines. Among the therapeutically useful proteins produced by mature hepatocytes include but are not limited to albumin, α1-antitrypsin, transferrin, blood coagulation factor VII, factor VIII, factor XII, protein C, and lipoproteins.

### BRIEF DESCRIPTION OF THE DRAWINGS

FIG. 1 is a bar graph showing the dose-response of hepatocytes on the proliferation of isolated ES-D3 cells on day six of culture. ES cells were cultured on collagen gel with no hepatocytes (control) or cocultured on top of collagen-sandwiched hepatocytes at low (12.5×10³ hepatocytes/cm²), medium (62.5×10³ hepatocytes/cm²), and high (125×10³ hepatocytes/cm²seeding densities. Relative cell growth of cultured ES-D3 cells was quantified by image analysis. Data shown are means±sd of two independent experiments in duplicate. *P<0.001. Similar results were reproduced in more than ten independent experiments for control and coculture with high hepatocyte density.

FIG. 2 presents biochemical analysis (6A) and animal survival curve (6B) of GalN-induced FHF rats connected to the BAL device for 10 hours, either with fibroblasts (3T3-J2) as a control, or with ESC-derived hepatocyte-like cells. The first dose of GalN (1.2 g/kg i.p.) was administered 24 hours after cannulation, and the second dose was given 12 hours later. The BAL perfusion started 24 hours after the first GalN injection. Total bilirubin (TBIL), blood urea nitrogen (BUN), aspartate transaminase (AST), and alanine transaminase (ALT). *P<0.05. Animal survival data using BAL device with primary porcine hepatocytes are from a published study (Shito et al., 111 J. Surg. Res. 111 53-62 (2003)).

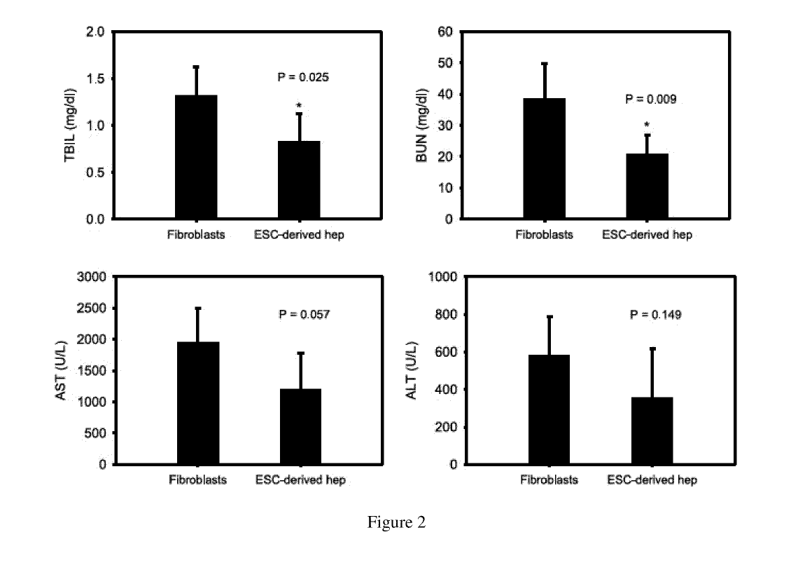

FIGS. 3A and 3B are bar graphs showing albumin and urea secretion rates of hepatocyte-like cells. Albumin secretion was measured at various timepoints by ELISA (3A). Urea secretion was measured with a colorimetric assay (3B). Only conditions incorporating DMSO-induction displayed a measurable level of albumin at day twenty-two. Urea and albumin secretion rates increased up to forty days of culture. No synergistic effect was observed with a combination of chemical inducers. Values represent mean±SD of two experiments and repeated in triplicate.

FIG. 4 is a bar graph presenting data from an EROD assay for cytochrome P450 activity. Day-thirty-four ES-derived cells were stimulated with 2 mM 3-methycholanthrene (3-MC) for two days. The conversion of ethyoxyresorufin to fluorescent resorufin was assayed on day thirty-six of culture. Values represent mean±SEM of three experiments and repeated in duplicate.

Color versions of these figures are presented in Cho et al., 22(3) FASEB J. 898-909 (2008), incorporated fully herein by reference.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The present invention provides for methods of generating mature functional hepatocytes from a defined endodermal population. Although extraembryonic tissue can express albumin and kidney tubular epithelium is able to produce urea, the molecular signatures and other identifying markers of these tissues would be divergent from the profile of hepatocyte differentiation. Replicating the developmental profile of pluripotent stem cells into mature hepatocytes confers reliability into the status of the differentiated hepatocytes. As a result, the PS cell-derived hepatocytes display high levels of hepatocyte function for long-term culture.

The pluripotent stem (PS) cell-derived hepatocyte precursors and mature hepatocytes may be used for direct cell therapy or utilized in an extracorporeal bioartificial liver device to treat liver failure. Additionally, PS cell-derived mature hepatocytes may replace isolated primary hepatocytes or hepatocarcinoma cell lines which are routinely used by the pharmaceutical industry for drug screening assays. Another area of application for the differentiated hepatocytes is in the production of therapeutically useful proteins.

The normally non-proliferative hepatocytes in the liver have a remarkable ability to regulate its growth mass. After a 30-60% partial hepatectomy, the rat liver can regenerate its original volume within two to three weeks (Michalopoulos et al., 276(5309) Liver Regeneration. Sci. 60-66 (1997)), while the human liver takes sixty days to regenerate (Wang et al., 100(S1) P.N.A.S. 11881-88 (2003)). Unfortunately this regenerative capacity is compromised with liver disease. Attempts to proliferate adult hepatocytes in vitro for reconstitution back into the diseased liver have not been successful. Although resident liver stem cells (also termed “oval cells”, “hepatocyte precursors”, or “hepatoblasts”) are present in rodents and show some capacity to proliferate and differentiated into mature hepatocytes (Wang et al., 2003; Rogler, 150(2) Am. J. Pathol. 591-602 (1997)), their existence in humans is not yet known. A method other investigators have suggested for proliferating hepatocytes is through cell immortalization by transfecting adult hepatocytes with the large T antigen of the SV40 virus (Pfeifer et al., 90(11) P.N.A.S. 5123-27 (1993).

Human bone marrow-derived stem cells have been shown to differentiate to hepatocytes in vitro (Jiang et al., 418 Nature, 41-49 (2002); Schwartz et al., 109 J. Clin. Invest. 1291-302 (2002)), and reverse liver failure in vivo (Jang et al., 6 Nat. Cell Biol. 532-39 (2004)). Both cell types are present in minute fractions, however, and thus are tedious to isolate and difficult to expand. Clearly alternative methods to generate mature functional hepatocytes must be established.

Pluripotent stem (PS) cells are capable of self-renewal and differentiation into most cell types in the body, and can theoretically provide a nearly limitless supply of differentiated cells. PS cells can be categorized in three different ways: embryonic stem (ES) cells, embryonic germ (EG) cells, and pluripotency-reprogrammed somatic cells. ES cell lines are derived from extracting cells of the epiblast in a developing blastocyst (Thomson et al., 282(5391) Science, 1145-47 (1998); Evans & Kaufman, 292(5819) Nature 154-56 (1981)), and EG cells are isolated from fetal gonadal tissue (Shamblott et al., 95(23) P.N.A.S. 13726-31 (1998); Matsui et al., 70(5) Cell, 841-47 (1992)). Somatic cell reprogramming for pluripotency is currently feasible in three different ways: somatic cell nuclear transfer (Wilmut et al., 385(6619) Nature, 810-13 (1997)), nuclear fusion of a somatic cell with ES cells (Cowan et al., 309(5739) Science 1369-73 (2005)), and transfection of pluripotency and proliferation genes into somatic cells (Takahashi & Yamanaka, 126(4) Cell 663-76 (2006)). Spontaneous differentiation of PS cells results in differentiation of a mixed cell population (teratoma) comprising cells of ectoderm, mesoderm, and endodermal lineage.

Development of protocols to induce endoderm from ES cells has severely lagged when compared with protocols for the ectodermal or mesodermal counterparts. As a result there is no adequate cell source for endodermal tissues derived from the embryonic stem cells. Advanced efforts to characterize endoderm in depth have suffered from several obstacles, including the lack of an endodermal cell line, scarcity of isolated endoderm from embryonic tissue, lack of control of differentiation, as well as the absence of any known cell surface markers for endoderm. Of the few available protocols available (Chang & Zandstra, 88(3) Biotechnol Bioeng, 287-98 (2004); Kubo et al., 131(7) Devel. 1651-62 (2004)), there are no reports of inducing a greater than 50% endoderm-enriched population, there are no reports of augmenting endodermal proliferation, nor are there any reports of controlling the endodermal population along one particular lineage.

More specifically, embryonic stem (ES) cells are considered a potential source of cells for hepatic therapies due to their limitless capacity for self-renewal and proliferation, and their ability to differentiate into all major cell lineages (Keller, 7 Curr. Opin. Cell Biol. 862-69 (1995); Odorico et al., 19 Stem Cells 193-204 (2001); Park et al., 7 Lab Chip 1018-28 (2007)). Several groups have already reported the differentiation of ES cells into hepatocyte-like cells. For example, ES cells cultured as embryoid bodies (EBs) differentiate spontaneously into hepatocyte-like cells (Chinzei et al., 36 Hepatology 22-29 (2002); Asahina et al., 9 Genes Cells 1297-1308 (2004); Jones et al., 272 Exp. Cell Res. 272,15-2216-20 (2002)). Efforts to induce a higher rate of differentiation toward the hepatic phenotype have shown limited success. These included various media and matrix combinations (Shirahashi et al., 13 Cell Transplant. 13, 197-211 (2004)), essential growth factors (Hamazaki et al., 497 FEBS Lett. 15-19 (2001)), compounds such as sodium butyrate (Rambhatla et al., 12 Cell Transplant. 1-11 (2003); Sharma et al., 94 Biotechnol. Bioeng. 1053-63 (2006)), or encapsulation-based systems (Maguire et al., 93 Biotechnol. Bioeng. 581-91 (2006); Maguire et al., 98 Biotechnol. Bioeng. 631-44 (2007)). The differentiation of ES cells toward the hepatic phenotype has resulted in mixed cell populations and low yields in the range of 10-50%.

One alternative approach for the differentiation of ES cells along the hepatic lineage is to expose them to cues from the liver. For example, recent studies have shown that stem cells from mesenchymal and hematopoietic origin can be induced to become hepatocyte- like cells following coculture with liver cells (Jang et al., 6 Nat. Cell Biol. 532-39 (2004); Lange et al., 11 World J. Gastroenterol. 4497-504 (2005)). One group has shown that immature ES cells can be driven to differentiate into a hepatic phenotype following transplantation in partially hepatectomized nude mice (Imamura et al., 10 Tissue Eng. 1716-24 (2004)). Another group has shown that ES cell engraftment into the liver can correct factor IX deficiency in mice. (Fair et al., 102 P.N.A.S. U.S.A. 102, 2958-963 (2005)). Taken together, these studies suggest that liver-specific cues may direct differentiation toward a hepatic phenotype both in vivo and in vitro.

The present invention provides for a rapid differentiation method to obtain a homogeneous endoderm-like cell population with 95% purity. The direct differentiation was achieved by culturing mouse ES cell on top of a collagen sandwich of primary rat hepatocytes. The presence of adult hepatocytes, but not liver endothelial cells or fibroblasts, promotes the differentiation and the proliferation of ES cells into a strikingly uniform population of endoderm-like cells, expressing the major endodermal markers Forkhead box protein A2 (Foxa2, formerly HNF3β), SRY-box containing gene 17 (Sox17), and alpha-fetoprotein (AFP) (Zaret 209 Dev. Biol. 1-10 (1999); Zaret, 3 Nat. Rev. Genet. 499-512 (2002)). When these ES cell (ESC)-derived endoderm-like cells were re-plated on a feeder layer of 3T3-J2 fibroblasts, further proliferation and differentiation along the hepatocyte lineage was observed, demonstrating hepatic morphology, functionality, and gene and protein expression. Furthermore, by seeding a bioartificial liver (BAL) device with these ES-derived hepatocyte-like cells we demonstrate an increased survival of rats following D-galactosamine (GalN)-induced fulminant hepatic failure (FHF).

The results of our studies indicate that the culture of mouse ES cell on top of collagen-sandwiched primary rat hepatocytes stimulates their differentiation and proliferation into a homogeneous population of endoderm-like cells. The endoderm-like cell population, which emerged within the first week of culture, had cubical cell morphology and bright cell borders and maintained a high level of gene and protein expression for the endoderm specific genes Foxa2, Sox17, and AFP (Zaret, 209 Dev. Biol. 1-10 (1999); Zaret, 3 Nat. Rev. Genet. 499-512, (2002)). This cell population was also negative for major mesoderm and ectoderm markers suggesting our system induced an endodermspecific differentiation. To our knowledge, this is the first demonstration of ES cell differentiation into endodermlike cells with a yield approaching 95%. This endoderm-like cell population could be further differentiated to hepatocyte-like cells, by subculturing the cells on a feeder layer of 3T3-J2 fibroblasts. The hepatocyte-like cell population that emerged had a hepatocyte morphology, expressed hepatocyte markers on the gene level, stained positive for albumin and CK-18 filaments, and secreted urea. In addition, these hepatocyte-like cells were shown to be efficacious in treating a rat model of GalN-induced FHF when seeded in an extracorporeal BAL device.

During development, the first sign of liver morphogenesis is a thickening of the ventral endoderm, which occurs around embryonic day eight in the mouse (Zaret, 11 Curr. Opin. Genet. Devel. 568-74 (2001)). Little is known about the signals involved in initial endoderm formation and subsequent endoderm specification, but recent studies suggest a role for FGF, BMP, and activin. FGF, both acidic and basic, produced by the cardiac mesoderm was shown to induce the foregut endoderm to the hepatic lineage (Zaret, 2001), while BMP produced by the transversum mesenchyme was shown to increase levels of GATA4 (Wells & Melton, 15 Annu. Rev. Cell Devel. Biol. 393-410 (1999)). Activin was also shown to participate in the early induction of endoderm through smad2 signaling (Ball & Risbridger, 238 Devel. Biol. 1-12 (2001)). By embryonic day 8.5 in the mouse, definitive endoderm has formed the gut tube and expresses Foxa2. As liver morphogenesis progresses, Foxa2 positive cells proliferate to form the hepatic diverticulum while expressing AFP. Final maturation of these hepatic progenitors occurs when hepatic cords associate with the mesenchymal cells of the septum transversum, forming the liver sinusoids while expressing albumin and urea (Zaret, 2001).

A similar path of differentiation occurs during culture of embryoid bodies (EB), which is the most common method of ES cell differentiation (Hamazaki et al., 497 FEBS Lett. 15-19 (2001)). Normally the early cell populations that arise during initial ES cell differentiation consist of neuroectoderm, mesoderm, definitive endoderm, and extraembryonic endoderm. The bipotent mesendoderm may also be present (Kubo et al., 131 Devel. 131,1651-1662 (2004); Tada et al., 132 Devel. 4363-74 (2005)). Although this mixed cell population does give rise to hepatocyte-like cells, usually in close proximity to cardiac-like tissue, the low yield makes it essential to purify the cell population to further explore their potential. In an effort to create a homogeneous cell population, several groups have studied endoderm differentiation in monolayer culture thereby exposing the ES cells to uniform cues from their microenvironment. One group has reported the presence of mesendodermal precursors in these cultures as well as the importance of collagen type IV to induce endoderm with a yield of up to 50%. Tada et al., 2005. This group and others have observed a characteristic epithelial morphology of Foxa2-positive endodermal cells in culture. Hisatomi et al., 39 Hepatology, 667-675 (2004). In addition to in vitro culture of ES cells, several groups have shown that liver cells and regenerating livers can induce hepatocyte-like gene expression in stem cells. Others have reported that liver cells can induce hepatic gene expression in cultures of mesenchymal and hematopoietic stem cells. Lange et al., 11 World J. Gastroenterol. 4497-504 (2005); Jang et al., 6 Nat. Cell Biol. 532-39 (2004). Similarly, it has been shown that the regenerative liver can drive ES cell differentiation into a hepatic phenotype. Imamura et al., 10 Tissue Eng. 1716-24 (2004).

Several groups have shown the importance of collagen in the differentiation of ES cells toward the hepatic phenotype. The differentiation of ES cells on three-dimensional collagen gels has not been previously investigated, however. The ability of three-dimensional collagen gels to induce and maintain epithelial morphology and function is well established, suggesting a similar enhancement of epithelial differentiation might occur during ES culture. Therefore, to create an environment conducive for hepatic differentiation we cultured ES cells on top of collagen-sandwiched hepatocytes. The uniform microenvironment, three-dimensional collagen gel, and cues from isolated hepatocytes contributed to the formation of a homogeneous cell population, which was 94.2% positive for Foxa2 and 95.9% positive for AFP.

Our group has previously shown that primary hepatocyte trapped between two layers of collagen gel (sandwich) maintain a high level of liver-specific function (Dunn et al., 3 FASEB J. 174-77 (1989); Dunn et al., 116 J. Cell Biol. 1043-53 (1992)). One explanation for the uniform differentiation observed in our coculture is activin secretion. Hepatocytes have been shown to secrete activin and other TGF-β family mitogens (Yasuda et al., 92 J. Clin. Invest. 1491-96 (1993)), which have been shown in numerous studies to promote endoderm induction (Kubo et al., 131 Devel. 1651-62 (2004); D'Amour et al., 23 Nat. Biotechnol. 1534-41 (2005)). Our results demonstrate that ES cells cocultured with hepatocytes rapidly lose the Oct-4 gene, which is important for the maintenance of pluripotency. However, coculture with isolated liver endothelial cells has failed to induce ES cell differentiation. This result is in agreement with in vivo studies suggesting that endothelial cells are important for the later stage of differentiation but not for the initial induction. Similarly, embryonic fibroblasts failed to cause a decrease in Oct-4 expression in ES cells. Mouse embryonic fibroblast feeders have been used to support the maintenance of undifferentiated ES cells. Conditioned media from the fibroblasts was also known to support the ES cell growth in undifferentiated state (Xu et al., 19 Nat. Biotechnol. 971-74 (2001)). Similar results were observed in this study.

In addition to differentiation, hepatocytes have also been shown to enhance the proliferation of various cell types (Endo et al., 40 J. Hepatol. 399-404 (2004); Skrtic et al., 20 Liver, 157-64 (2000); Gressner et al., 13 Liver, 86-94 (2004)). In a similar fashion, our results demonstrate that hepatocytes stimulate ES cell proliferation in a dose-dependent fashion due to secreted factors. Similar increase in ES cell proliferation was also seen when the cells were separated by transwells, although the proliferation rate was decreased. In both cases of differentiation or proliferation in coculture the ES cells were separated by more than 400 μm from the hepatocytes and were never in contact, there-fore cell fusion and cell-cell contact could be ruled out. Genetic characterization of cocultured ES cells for gender-specific DNA sequences demonstrated that the differentiating male ES cells were not fusing with female rat hepatocytes.

The endoderm-like population that emerged in our studies had the characteristic epithelial morphology of Foxa2-positive endodermal cells, which was previously demonstrated (Tada et al., 132 Devel. 4363-74 (2005); Hisatomi et al., 39 Hepatology, 667-75 (2004)). Spindle-like, elongated, or elliptical cells were not observed, and the gene expression data demonstrated endodermal but not mesodermal (Gata-1, Runx2, Foxf1, Nkx2.5, Tal1) or neuroectodermal (Pax6) markers. These findings suggest that the enhanced proliferation and differentiation observed in our system was lineage-restricted to endoderm. The gene expression panel and cellular morphology were similar on day six and day ten of coculture, suggesting that the proliferation was symmetric, and not asymmetric. This, however, needs to be carefully studied using single cell lineage tracing and mapping techniques.

Previous studies have demonstrated the importance of FGF, HGF, oncostatin-M, and cortisone on the differentiation of ES cells into hepatocyte-like cells. Hamazaki et al., 497 FEBS Lett. 15-19 (2001). These factors are commonly secreted by the cardiac mesoderm and the mesenchymal cells. To induce the late-stage hepatic differentiation we chose to culture the endoderm-like cells on growtharrested 3T3-J2 fibroblasts. Fibroblasts are known to produce a variety of soluble growth factors and ECM components, including FGF, HGF, and proteoglycans (Story, 25 In Vitro Cell Devel. Biol. 25, 402-08 (1989); Matsumoto et al., 188 Biochem. Biophys. Res. Commun. 235-43 (1992); Schmidtchen et al., 265 Biochem. J. 289-300 (1990)). We found that this system provided the best survival and proliferation of the hepatocyte-like cells, when compared to cultures of these cells on single gel, double gel, and matrigel. The ESCderived endoderm-like cells continued to proliferate and differentiate into a uniform population of hepatocyte-like cells, which form epithelium-like clusters. Some binucleated cells were observed in the colony clusters. Previous reports have shown that endodermal cells alone fail to differentiate and mature (Tada et al., 2005; Ishii et al., 309 Exp. Cell Res. 68-77, (2005)), suggesting that significant mesenchymal/mesodermal cues are necessary for the maturation of the early endodermal cells (Wells & Melton, 127 Devel. 1563-72 (2000)). Taken together, these studies suggest that direct cell-cell contact with mesenchymal or mesodermal cells provides a suitable environment for endoderm proliferation and differentiation, consistent with our studies.

We have previously shown that 3T3-J2 fibroblasts promote a high level of hepatic gene expression, protein expression, and function in isolated hepatocytes (Bhatia et al., 13 FASEB J. 1883-1900 (1999)). Similarly, our results demonstrate a strong activation of the hepatic genes by day 28 of culture, stained positive for albumin and CK-18, and secreted urea. Morphologically the cells appear hepatocyte-like, including many binucleated cells, dense cytoplasm, and bile canaliculi. Optimization of the culture conditions for terminally differentiating the hepatocyte progenitor cells into mature hepatocytes resulted in albumin and urea secretion levels similar to those seen with rat primary hepatocytes (unpublished data). Although urea production and albumin expression are characteristic of hepatocyte activity, kidney tubular epithelium also produces urea (Dunn et al., 7 Biotechnol. Prog. 237-45 (1991)), while extraembryonic cells express albumin. To our knowledge only hepatocytes do both, which suggests that the cells attain a hepatic phenotype. Further studies provide characterization of these cells at a molecular level.

A means of further evaluating the functional capacity of the ESC-derived hepatocyte-like cells is to determine their therapeutic efficacy in treating FHF. Rats with GalN-induced FHF received a 10 hour perfusion with a BAL device seeded with the ESC-derived hepatocytelike cells. Plasma levels of liver enzymes were reduced in the animals treated with the device containing the hepatocyte-like cells compared to the animals treated with the device containing fibroblasts. Animal survival on day five was significantly increased compared to the animals receiving treatment with a BAL device seeded with fibroblasts. Comparing these results to those from our prior study using primary porcine hepatocytes in the BAL device to treat FHF in the same rat model revealed similar survival trends. This suggests that the ESC-derived hepatocyte-like cells were providing biochemical support to the animals that was similar to primary hepatocytes, and resulted in increased animal survival. Also, it may be possible to increase the liverspecific functions of the ESC-derived hepatocyte-like cells in the BAL device by coculturing them with nonparenchymal cells, such as fibroblasts or endothelial cells, as seen with primary hepatocytes. Current studies explore the in vivo therapeutic efficacy of the ESC-derived hepatocyte-like cells.

More specifically, primary rat hepatocytes cultured in a collagen-sandwich configuration (Dunn et al., 3 FASEB J. 174-77 (1989); Dunn et al., 116 J. Cell Biol. 1043-53 (1992)), have been shown to maintain a high level of liver-specific function and to secrete numerous proteins. To determine the ability of collagen-sandwiched rat hepatocytes to induce ES cell differentiation, the cells were seeded on top of the collagen sandwich. The 400-μm-thick collagen layer did not permit direct hepatocyte-ES cell contact.

Adult hepatocytes stimulate ES cell differentiation and proliferation in cocultures. Octamer-4 (Oct-4) is an important marker of pluripotency, expressed by primitive embryonic cells both in vivo and in vitro (Niwa et al., 24 Nat. Genet. 372-76 (2000)). Oct-4 is downregulated during early differentiation. To monitor early differentiation in our system, we studied the ES-R1 mouse ES cell line in which green fluorescent protein (GFP) was knocked in to monitor Oct-4 gene expression. To assess the effect of coculture on ES cell differentiation, we monitored Oct-4 gene expression following five days of coculture with either rat liver parenchymal cells (adult rat hepatocytes), rat liver sinusoidal endothelial cells (LSEC), or mouse embryonic fibroblasts (MEF). Using flow cytometry analysis, we observed that Oct4-GFP expression of ES cells cultured on top of collagen sandwiched hepatocytes was decreased significantly compared with the other three culture conditions on day five of culture. ES cells cultured on top of collagen-sandwiched embryonic fibroblasts or liver endothelial cells maintained strong Oct4-GFP expression, forming Oct4-GFP positive aggregates. Similar results were observed in direct cocultures of Oct4-GFP ES cells on feeder layers of fibroblasts, endothelial cells, and hepatocytes in the absence of collagen gel layer. These results indicate that primary rat hepatocytes, but not endothelial cells or fibroblasts, stimulate the differentiation of ES cells in collagen culture.

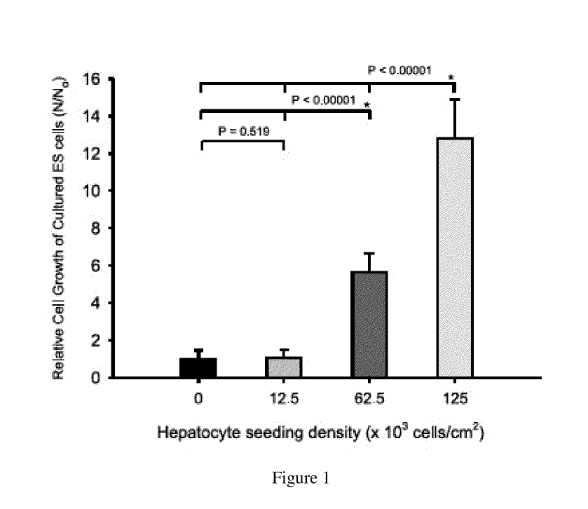

One possible route by which coculture can induce ES cell differentiation is by altering the growth kinetics of the ES cells. When cocultured with hepatocytes the ES cells underwent rapid proliferation without forming Oct4-GFP positive aggregates. One possibility is that a hepatocyte-secreted soluble factor is responsible for the increased proliferation. To evaluate the effect of hepatocytes on the proliferation of ES cells (ES-D3), we seeded a constant number of ES cells (6.25×10³ cells/cm²) on collagen-sandwiched hepatocytes seeded at low (12.5×10³), medium (62.5×10³), and high (125×10³ hepatocytes/cm²) seeding densities. A hepatocyte “dose-dependent” increase in the area covered by ESC-derived cells was observed on day 6 of culture (FIG. 1). We verified that the area per individual ESC-derived cell was constant (165±28 μm² in area), confirming that the increasing coverage correlates directly with an increase in cell number. Similar to the Oct4-GFP ES cells (derived from ES-R1 cells), ES-D3 cells cultured on top of collagen-sandwiched hepatocytes showed a significantly higher proliferation rate when cultured with hepatocytes at medium and high densities compared to those in single culture (P<0.00001). No significant differences were observed between ES cells in single culture and ES cells cocultured with hepatocytes at low density (P=0.519). The data show that ES cell proliferation increased as a function of hepatocyte density. These results indicate that primary rat hepatocytes stimulate the proliferation of ES cells in collagen culture.

ES cells cocultured with adult hepatocytes differentiate into a homogeneous population of endoderm-like cells. To study the differentiation of ES cells in our coculture system we analyzed gene and protein expression on days six and ten of coculture. Using RT-PCR we observed that the ES cells cocultured with hepatocytes clearly expressed higher levels of the endoderm markers Foxa2, Sox 17, and AFP on day six than the ES cells in single culture. The mesendodermal markers Brachyury and Goosecoid (Kubo et al., 131 Devel. 1651-62 (2004); Tada et al., 2005), were upregulated on day four of differentiation and downregulated rapidly on days six and ten, indicating a transient mesendodermal population. Phase contrast image of the proliferating ESC-derived cells in day ten of coculture with adult hepatocytes shows a uniform and homogeneous population of ESC-derived cuboidal cells with bright cell-cell borders, reminiscent of isolated endodermal cells (Tada et al., 2005; Hisatomi et al., 2004). The ESC-derived cells were labeled with BrdU, a DNA synthesis marker, demonstrating that the cells retain their ability to proliferate.

Further characterization of the ESC-derived cells on day ten by immunostaining demonstrated high level of Foxa2 and AFP protein expression. In addition, flow cytometry analysis of the ESC-derived population demonstrated that 94.2±0.3% of the cells were Foxa2-positive, and 95.9±0.8% of the cells were AFP-positive, demonstrating the formation of a homogenous endoderm-like population. Since ectoderm, endoderm, and mesoderm may all be present in early ES cell differentiation schemes, we determined whether key markers for these cell types were expressed in our culture conditions. We chose a combination of early transient markers and markers for ectoderm, endoderm, and mesoderm. Using RT-PCR, we examined the expression of the liver transcription factors Foxa2 (HNF-3β, required for endoderm specification) (Ang et al., 119 Devel. 1301-15 (1993); Nagy et al., 126 J. Cell Biol. 223-33 (1994)) and Sox 17 (an endoderm marker), alphafetoprotein (AFP, an endoderm and early hepatocyte marker), albumin (a hepatocyte marker), GATA1 (hematopoietic cells), Runx2 (mesenchymal cells), Foxf1 (mesenchymal cells), Nkx2.5 (cardiac cells), Tal1 (endothelial cells), and Pax6 (an ectoderm marker) of the ES cells cultured on top of collagen-sandwiched adult hepatocytes for six and ten days. We found that the differentiating ES cells cultured on top of collagen sandwiched adult hepatocytes expressed Foxa2, Sox17, and AFP on days six and ten, but not albumin, suggesting that the cells differentiated into early endodermal cells. The mesodermal and ectodermal markers were not expressed on culture days six and ten, suggesting that the cells in this culture condition were endoderm-derived.

To rule out the possibility of cell fusion between the differentiating mouse ES cells and the female rat hepatocytes, we performed a simple genotype analysis using gender determination of the cells. Our analysis demonstrated that the undifferentiated ES cells (day 0) and the cocultured ES cells after ten days of differentiation had double bands (XY genotype) with identical magnitude indicating that they were from male animals. Rat hepatocytes used for ES cell coculture displayed a single band indicating that the animal was female. These results show that the differentiating ES cells in coculture were not fusing with rat hepatocytes.

ES cell-derived endoderm-like cells can differentiate into hepatic lineage cells. During development, endoderm-derived hepatic precursors associate with mesenchymal cells of the septum transversum before fully maturing into functional hepatocytes. Therefore, in order to differentiate the ES cell-derived endoderm-like cells, we reseeded the cells on top of growth arrested fibroblast (3T3-J2) feeder layers at a density of 6.25×10³ ES cells/cm². Following twenty-eight days in culture, the cells formed epithelial-type clusters and appear as a uniform population with morphology that is very similar to that of adult hepatocytes. Some binucleated cells were observed in the colony clusters at late stage of culture (see inset). Although similar morphology was seen when the cells were cultured alone on collagen gel, cellular proliferation was significantly retarded. To further characterize the phenotype of these ESC-derived hepatocyte-like cells, we studied gene and protein expression using immunofluorescence and RT-PCR. The ES cell-derived hepatocyte-like cells were positive for the mature hepatic markers, albumin and CK-18, on the protein level (day twenty-eight). Hepatocyte gene expression of the ESC-derived hepatocyte-like cells was upregulated on days eighteen and twenty-eight. The ES cell-derived hepatocyte-like cells were positive for hepatocyte markers, including albumin, alpha-l-antitrypsin (AAT), CK-8, CK-18, transthyretin (TTR), and cytochrome P450 3A13 (CYP3A13). Foxa2 was weakly expressed on days eighteen and twenty-eight, similar to gene expression of mature hepatocytes. AFP, an endodermal and early hepatic marker, was upregulated on day twenty-eight, however, suggesting that some of the cells may still be in early stages of hepatic lineage. In addition, the high level of AFP expression on day twenty-eight might be due to the proliferation of hepatic progenitors. No expression of mesodermal or ectodermal markers was detected in any of these cultures. We also investigated urea synthetic ability of differentiating ESC-derived cells to assess hepatocyte-specific function. The activity of urea synthesis on day eighteen was 6.1 μg/48 h/35 mm dish. The level of urea production increased to 17.9 μg/48 h/35 mm dish in late stage of culture (day twenty-eight). These results indicate that the ESC-derived endodermlike cell population could be further differentiated to hepatocyte-like cells.

Biochemical analysis and animal survival from liver failure was undertaken to evaluate the therapeutic efficacy of the ES cell-derived hepatocyte-like cells. We attempted to rescue rats undergoing GalN-induced FHF by incorporating the hepatocyte-like cells in a BAL device and treating for ten hours. We measured changes in liver enzymes [aspartate transaminase (AST), alanine transaminase (ALT)], total bilirubin (TBIL), and blood urea nitrogen (BUN) immediately following the ten hours extracorporeal perfusion. Significant reductions were found in the plasma levels of TBIL and BUN (P=0.025, P=0.009, respectively), and trends were noted for decreased AST and ALT levels (P=0.149, P=0.057, respectively) in the animals treated with the BAL device containing ESC-derived hepatocytes compared to the control group.

Animal survival was evaluated up to 120 hours (five days) after the first GalN injection. In the animal group treated with the BAL device seeded with the hepatocyte-like cells, five out of six animals (83.3%) survived at 48 hours compared to three out of eight (37.5%) rats that were treated with a BAL device seeded with fibroblasts (control group). By day five, three out of six (50.0%) rats survived in the ESC-derived hepatocyte seeded BAL group compared to one out of eight (12.5%) rats in the control group. The differences between the two groups were significant on day five (P=0.034) using the generalized Wilcoxon's test. Animal survival from the ESCderived cell seeded BAL group was also compared to our previously published results in which rats with GalN-induced FHF receiving treatment with a BAL device containing primary porcine hepatocytes had 58.3% survival on day five (Shito et al., 2003). Similar results in animal survival were observed in both groups through day five.

A transgenic ES cell for enhanced green fluorescence protein (GFP) driven by the albumin promoter was used or the purpose of quickly screening different stimulants for hepatic differentiation. After a total of 30 days of differentiation, the various agonists were evaluated for their ability to induce ES-derived endoderm (ESDE) to express the albumin gene. When the ESDE cells were cultured in high glucose conditions, none of the agonists tested were able to increase cell population fraction positive for GFP expression over 10% (Table 1):

TABLE 1
Cell fraction of day 30 ES-derived cells
positive for albumin promoter-driven GFP expression

| | High Glucose | Low Glucose |
|---|---|---|
| 2% DMSO | 3.8 | 38 |
| 1 μM Azacytidine | 9.5 | 29 |
| 1 μM Retinoic Acid | 2.9 | 29 |
| 100 μM SNAP | 6.2 | 48 |
| 1 μM A23187 | 7.4 | 41 |
| 1 nM vasopressin | 4.5 | 59 |
| 200 μM IBMX | 5.8 | 43 |
| 2 mM phenobarbital | 4 | 44 |
| 10 mM nicotinamide | 5.6 | 43 |
| 0.1 μM PGE2 | 7.8 | 57 |
| control | 7.4 | 32 |

Under low glucose culture conditions, however, all of the agonists were able to increase the percentage of cells that were positive for GFP fluorescence to at least 29%. Dimethyl sulfoxide (DMSO), all-trans retinoic acid, S-nitroso-N-acetylpenicillamine (SNAP), nicotinamide, and prostaglandin E2 (PGE2) stimulation resulted in 38%, 29%, 48%, 43%, and 57% GFP-positive fraction, respectively. Non-stimulated ESDE cells in low glucose culture had 10% of its population positive for GFP after thirty days of culture. One (1) μM 5′-azacytidine and 1 μM Ca⁺² ionophore A23187 displayed an approximately 30% and 40% GFP-positive fraction, respectively; while 2mM phenobarbital and 0.2 mM 3-isobutyl-1-methylxanthine (IBMX) treatment resulted in ˜44% GFP-positive fraction.

ESDE cells did not express any of the genes characteristic of mature hepatocytes at day fourteen. After agonist induction of differentiation with DMSO or RA, expression of albumin (Alb) and cytochrome P450 subunit 3a13 (Cyp3a13) mRNA could be detected by day twenty-two and increased expression was found at day thirty. Transcriptional expression of liver-specific enzyme tyrosine aminotransferase (Tat) was also found to increase with culture time and was highest at day thrity only with DMSO or RA induction. Similarly, glucose-6-phosphatase (G6pc) mRNA expression could only be detected with DMSO or RA stimulation. Definitive endoderm marker Sox17 was observed in all conditions but appeared to be down-regulated with DMSO induction. Visceral endoderm marker Sox7 was also weakly expressed in every condition except with DMSO induction. Although alpha-fetoprotein (AFP) and albumin are found in visceral endoderm as well as fetal hepatocytes, the absence of Sox7 transcripts combined with the presence of mature hepatocyte markers suggests that DMSO stimulation of ESDE cells confers differentiation of hepatocyte precursors and mature hepatocytes.

After induction of differentiation of the ESDE cells with DMSO in low glucose media, we monitored the maturation of the cells into hepatocytes. Combined immunofluorescence of alpha-fetoprotein and albumin protein expression was used to observe the transition of hepatic progenitors into mature cells. At twenty-six days of culture, the ESDE cells adopted a fibroblastic or mesenchymal cell-like morphology. While most of the cells stained for both AFP and albumin, the pattern of protein expression appeared to be inversely correlated. The smaller cells displayed stronger expression of AFP but the flatter epithelial-like cells showed higher levels of albumin.

By day thirty-two of culture, the morphology of the ESDE cells transformed into flat epithelial cells, many of which were bi-nucleated. Bright, definable cell borders were observed at the inter-cell junctions, characteristic of primary hepatocytes cultured in vitro. Many of the cells also co-expressed mature hepatocyte proteins cytokeratin-18 and albumin.

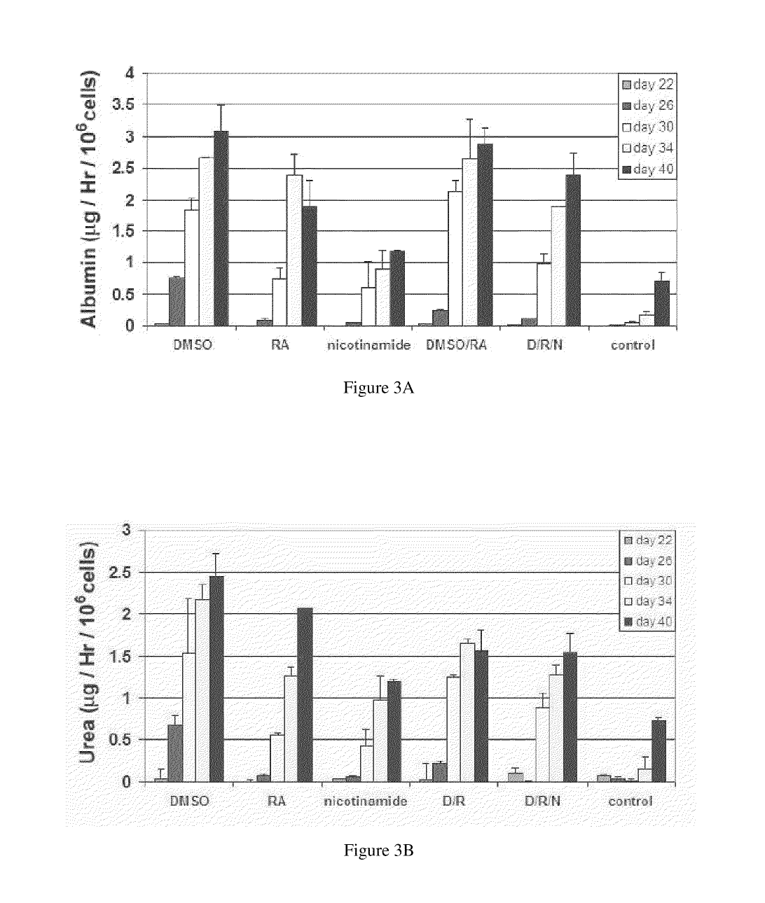

The maturation of ESDE cells into functional hepatocytes was evaluated by measuring the secretion of albumin and urea into the culture media (FIG. 3). With DMSO induction, albumin secretion was first detected at 0.34 μg/hour/10⁶ cells on day twenty-two, and the secretion rate increased steadily with culture time up to 3.09 μg/hour/10⁶ cells by day forty. DMSO-stimulated ESDE cells were capable of secreting urea at a rate or 0.36 μg/hour/106 cells on day 22, which increased to 2.45 μg/hour/10⁶ cells by day forty. Combining differentiation agonists did not appear to enhance the secretion rate of either albumin or urea. Neither albumin nor urea secretion was detectable before day twenty in any of the other conditions tested.

Rat primary hepatocytes are capable of secreting albumin at a rate of approximately 2.0 μg/hour/10⁶ cells, and the mouse hepatocarcinoma cell line Hepal-6 can produce albumin at a rate of 4.7 μg/hour/10⁶ cells. The ability of the ES-derived hepatocyte-like cells to produced and store glycogen was assessed using a periodic acid-Schiff stain. After thirty-two days of differentiation, the DMSO-stimulated ESDE cells showed visible glycogen stores. Many of the cells that were stained positive for glycogen were bi-or multi-nucleated, and possessed hepatocyte-like epithelial morphologies. The capacity for the hepatocyte-like cells to actively synthesize glycogen was seen after the culture media was switched from the low-glucose differentiation media to high-glucose DMEM supplemented with 10% FBS and 0.2 μM dexamethasone for two days. More intense magenta staining of the hepatocyte-like cells was found after the two-day incubation in high-glucose media.

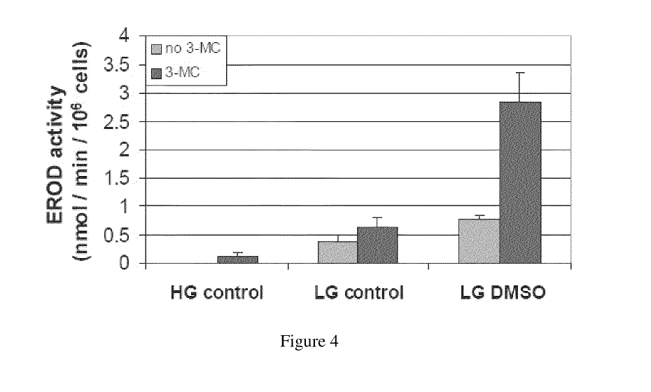

Xenobiotic metabolism is an important function of the liver. The EROD assay was used to measure inducible cytochrome P450 activity (CYP1A1) of the hepatocyte-like cells. After a two-day exposure of 3-methylcholanthrene to the DMSO-induced hepatocyte-like cells displayed a 4.5-fold greater level of EROD activity over cells cultured in low glucose media without DMSO stimulation (FIG. 4). EROD activity of the DMSO-induced hepatocyte-like cells was also 20-fold higher compared to cells cultured in high glucose media without DMSO stimulation.

In conclusion, the present invention provides for the derivation of functional hepatocytes from embryonic stem cells. An ES cell culture protocol capable of generating ˜95% positively expressing Foxa2 and alpha-fetoprotein endoderm-like cells by co-culturing murine ES cells on top of collagen gel sandwiched rat hepatocytes for ten days. ES cell hepatic differentiation was initiated by re-seeding the d10 ES cell-derived endoderm-like (ESDE) cells on collagen gel, followed by a six-day induction period (d14-d19) with 2% dimethyl sulfoxide (DMSO) or 1 μM all-trans retinoic acid (RA) in low glucose (1 g/L) DMEM. DMSO induction resulted in a down-regulation of the endodermal gene Sox17, while an up-regulation of hepatocyte genes tyrosine aminotransferase, glucose-6-phosphatase, and albumin was observed. By day thirty, many of the differentiated ES cells were mono- and bi-nucleated epithelial cells co-expressing cytokeratin-18 and albumin. After thirty days of differentiation with DMSO or RA induction, albumin and urea secretion rates of the ESDE cells were similar to the secretion rates of primary rat hepatocytes. Active glycogen synthesis and drug metabolizing activities were also observed with the ES cell derived hepatocyte-like cells.

The production of a hepatic progenitor cell population may lead to a variety of new products. In theory, a committed progenitor, such as a hepatic progenitor cell could be generated from the endoderm and used for cell therapies to treat acute or chronic liver failure as well as for further maturation and confinement within a tissue-engineered extracorporeal BAL device. Similarly, new generations of other cellular products and tissue-engineered products could be designed using an ES cell-derived endodermal cell. A reproducible culture system as described herein yields a unique and homogeneous population of endoderm-like cells that can be further differentiated into hepatic lineages without losing their characteristics. These populations derived from ES cells have the potential to become a reliable source of cells for in vivo cell transplantation, toxicology screens, and the development of BAL devices.

Additionally, current recombinant protein production is a significant technology which has given clinicians and researchers tools for treatments and understanding diseases. The two principal cells that are used in this field are the E. coli bacterium and the Chinese hamster ovary (CHO) cell line. Genetically engineered E. coli can produce simple proteins but lack the synthetic apparatus for post-translational modifications such as glycosylation and phosphorylation. Proper placement of these post-translational modifications is essential for the physiological activity of many proteins. CHO cells have most of the sub-cellular structures and enzymes to perform the post-translational modifications on proteins but they do lack certain enzymes such as 2,6-sialyltransferase. It is also not necessarily certain that the glycosylation and phosphorylation modifications are being performed at the correct locations along the protein sequence. The PS cell-derived hepatocytes of the present invention have the sub-cellular structures, enzymes, and pathways developed to produce the complex proteins and lipoproteins hepatocytes normally generate.

Furthermore, the cell culture methods the present invention may be used to screen a compound for its effects on a hepatocyte or on a hepatocyte activity. For example, a compound in question may be contacted with an embryonic stem cell differentiated along a hepatocyte lineage by culturing as shown herein, and determining any change to the cells or in an activity of the cells, then correlating such change with the effect of the compound. Examples of other non-therapeutic uses of the hepatocyte-like cells of the present invention include research of liver embryology, liver cell lineages, and differentiation pathways; gene expression studies including recombinant gene expression; mechanisms involved in liver injury and repair; research of inflammatory and infectious diseases of the liver; studies of pathogenetic mechanisms; and studies of mechanisms of liver cell transformation and etiology of liver cancer.

### Examples

Example 1

### Cell Cultures

Hepatocytes were isolated from adult female Lewis rats (Charles River Laboratories, Boston, Mass., USA) weighing 150-200 g, using a modified two-step collagenase perfusion procedure (Seglen, 13 Methods Cell Biol. 29-83 (1976)), as described previously (Dunn et al., 3 FASEB J. 174-77 (1989)). Primary rat hepatocytes were sandwiched between two layers of collagen to maintain their stable liver specific functions (Berthiaume et al., 10 FASEB J. 1471-84 (1996); Dunn et al., 116 J. Cell Biol. 1043-53 (1992)). Tissue culture dishes (35 mm) were coated with 0.5 mL of a mixed solution of 9 parts of type I rat tail collagen (1.1 mg/mL in 1 mM HCl) and 1 part 10× DMEM and incubated for 1 h at 37° C. to form a collagen gel. After gelation, 1 million hepatocytes (12.5×10³ cells/cm²) in 1 mL hepatocyte culture medium were seeded and incubated in 90% air/10% CO₂ at 37° C. To achieve uniform densities, the substrates were shaken every 15 min for the first hour after cell seeding. The following day, the culture medium was removed and a second collagen gel layer was overlaid on the hepatocytes and incubated for 1 h at 37° C. After gelation, 1 mL of hepatocyte culture medium was applied. Culture medium was changed daily. Hepatocyte culture medium consisted of DMEM supplemented with 10% fetal bovine serum (Life Technologies, Inc., Gaithersburg, Md., USA), 7 ng/mL glucagon (Bedford Laboratories, Bedford, Ohio, USA), 7.5 μg/mL hydrocortisone (Pharmacia Corporation, Kalamazoo, Miss., USA), 0.5 U/mL insulin (Eli Lilly, Indianapolis, Ind., USA), 20 ng/mL epidermal growth factor (Sigma Chemical Co., St. Louis, Mo., USA), 200 U/mL penicillin, and 200 μg/mL streptomycin (Life Technologies, Inc.).

The murine ES cell line D3 (ATCC, Manassas, Va., USA) was used to assess the differentiation of ES cells into endodermal and hepatic lineage cells. The murine Oct4-GFP ES cell line R1 (Mount Sinai Hospital, Toronto, ON, Canada) was used to monitor the early differentiation of ES cells. Undifferentiated ES cells were cultured in Knockout DMEM (Life Technologies, Inc.), supplemented with 15% replacement serum, 4 mM L-glutamine (Cambrex, Walkersville, Md., USA), 100 U/mL penicillin (Life Technologies, Inc.), 100 U/mL streptomycin (Life Technologies, Inc.), 10 μg/mL gentamicin (Life Technologies, Inc.), 1000 U/mL Leukemia Inhibitory Factor (LIF; Chemicon International, Temecula, Calif., USA), and 0.1 mM 2-mercaptoethanol (Life Technologies, Inc.) on gelatin-coated T75 tissue culture flasks. Culture medium was replaced every day, and cells were passaged at dilutions that ranged from 1:5 to 1:20 at least once a week. Cells from passage numbers 18-25 for ES-D3 and 26-29 for Oct4-GFP ES cells were used in the experiments. Cells were cultured at 37° C. and in a 95% air/5% CO₂ atmosphere. Unless otherwise noted, ES cells were seeded at a density of 6.25×10³ cells/cm² and cultured as monolayers in hepatocyte culture medium, which was changed daily. ES cells were seeded either on top of collagen-sandwiched hepatocytes, liver sinusoidal endothelial cells, or murine embryonic fibroblasts. Controls were established by seeding ES cells on top of a single collagen gel.

Additionally, for the purpose of quickly screening different stimulants for hepatic differentiation, a transgenic ES cell for enhanced green fluorescence protein (GFP) driven by the albumin promoter was used for these preliminary studies. The transgenic murine J1 cell line was obtained from the National Cancer Center Research Institute (Tokyo, Japan). The murine ES cell line D3 (ATCC, Manassas, Va.) was also used to assess the differentiation of ES cells into endoderm and hepatic lineage cells. Undifferentiated ES cells were cultured in Knockout DMEM (Gibco), supplemented with 15% replacement serum, 4 mM L-glutamine (Gibco), 100 U/mL penicillin (Gibco), 100 U/ml streptomycin (Gibco), 10 μg/mL gentamycin (Gibco), 1000 units/mL Leukemia Inhibitory Factor (LIF; Chemicon International, Temecula, Calif.), and 0.1 mM 2-mercaptoethanol (Gibco) on 0.1% gelatin-coated T-75 tissue culture flasks. Culture medium was replaced every day and cells were passaged at dilutions ranging from 1:5 to 1:20 at least once a week. Cells were cultured at 37° C. and in a 5% CO₂ incubator.

### Example 2

### Isolation and Culture of ES-Derived Cells

To separate the ES-derived cells from the hepatocytes in coculture, the cells were treated on day ten with 1 mg/mL dispase (Life Technologies, Inc.) for 60 min at 37° C. After the dispase treatment, the ES cell-derived cells could be detached by gentle pipetting without disturbing the collagen gel. The collagen-sandwiched hepatocytes remained at the bottom of the dish, thus allowing separation of the ES-derived cells from the primary hepatocytes. The ES-derived cells were then treated with trypsin to achieve single-cell suspension. The purified ES-derived cells were either analyzed for gene and protein expression or were replated in 35-mm culture dishes at a density of 50,000 cells per dish (6.25×10³ ES cells/cm²). For further differentiation, isolated ES-derived cells were plated either on collagen gels or growth-arrested 3T3-J2 fibroblast feeder layers and were cultured in hepatocyte culture medium supplemented with 100 ng/mL oncostatin-M (Sigma), 10⁻⁷ M dexamethasone (Sigma) and insulin/transferrin/selenious acid (5 μg/mL, 5 μg/mL, and 5 ng/mL, respectively) (BD Biosciences, San Jose, Calif., USA). To rule out the possibility of cell fusion occurring during the coculture process, we performed a simple genotype analysis of the cocultured ES cells on day ten. Because the rat hepatocytes were of female origin and the ES cells were male, we used a polymerase chain reaction (PCR)-based gender identification approach published previously (Cathey et al., 52 Evolution 1224-29 (1998); Shaw et al., 84 J. Mammal. 123-28 (2003)), to analyze the DNA of the cocultured population. This technique uses primers for conserved zinc finger regions of the X and Y chromosomes of the DNA of interest. We reasoned that if appreciable cell fusion had occurred, the XY chromosome analysis of cocultured ES cells would appear similar to the male XY genotype for undifferentiated mouse ES cells rather than the XX profile for female rat hepatocytes.

### Example 3

### Differentiation of ES-Derived Cells

To isolate the ES-derived cells in co-culture, the ES cells cultured on top of collagen sandwiched hepatocytes were incubated with dispase (1 mg/mL) (Gibco) for 30-60 min at 37° C. After incubation, the ES-derived cells were detached by gentle pipetting. The collagen-sandwiched hepatocytes remained at the bottom of the dish, thus allowing separation of the ES-derived cells from the primary hepatocytes. The isolated ES-derived cells were replated (day 10) on collagen gel coated 35-mm dishes at a density of 400,000 ES-derived cells per dish and cultured in high glucose DMEM supplemented with 10% FBS, 100 ng/mL oncostatin-M (Sigma), 0.2 μM dexamethasone (Sigma), and insulin/transferrin/selenious acid (5 μg/mL, 5 μg/mL, 5 ng/mL, respectively) (BD Biosciences, San Jose, Calif.). The replated cells were allowed to recover for four days before induction of hepatocyte differentiation.

At day fourteen of culture, the ES cells were stimulated with the chemical agonists listed on Table 1 (above) in high glucose or low glucose DMEM supplemented with 10% FBS and 0.2 μM dexamethasone. The media with chemical agonists were exchanged every other day until day twenty. At day twenty of culture, the ES cells were cultured without the chemical agonists in high glucose or low glucose DMEM supplemented with 10% FBS and 0.2 μM dexamethasone. The ES cells were cultured up to day fifty with media exchange every other day.

### Example 4

### Experimental Animals

Male Sprague-Dawley rats (Charles River Laboratories, Boston, Mass., USA), weighing from 250 g-350 g, were used for this study. All animals were acclimated to the animal research laboratory for 5 days prior to experiments and were maintained in a light-controlled room (12-h light-dark cycle) at an ambient temperature of 25° C. with chow diet and water ad libitum. These rats were maintained in accordance with National Research Council guidelines, and the experimental protocols were approved by the Subcommittee on Animal Care, Committee on Research, MA General Hospital.

### Example 5

### Surgical Procedures and Induction of FHF

Surgical procedures and induction of FHF are described in detail elsewhere (Shito et al., 2003). Briefly, male Sprague-Dawley rats (250 g-350 g) were anesthetized with an intraperitoneal (i.p.) injection of ketamine (Abbott Laboratoies, N. Chicago, Ill., USA) and xylazine (Phoenix Pharmaceuticals, St. Joseph, Mo., USA) at 110 and 0.4 mg/kg, respectively. The carotid artery and jugular vein were cannulated with 40 cm lengths of PE 50 polyethylene tubing (Becton Dickinson, Sparks, Md., USA) through a dorsal incision. The wound was sutured. The animal was transferred into a cage and was fasted until the first d-galactosamine (GalN; Sigma) injection. To prevent blood clotting, heparinized (20 U/mL) saline solution was continuously infused at a rate of 0.2 mL/h through the arterial line by a syringe infusion pump (Fisher Scientific, Pittsburgh, Pa., USA) until the extracorporeal perfusion experiments were initiated. GalN was freshly dissolved in 0.5 mL of physiological saline and adjusted to pH 6.8 with 1 N NaOH. The first dose of GalN (1.2 g/kg i.p.) was administered 24 h after cannulation, and a second dose was given 12 h later. After the first injection, the rats had free access to food and water until sacrifice.

### Example 6

### BAL Device and Cell Seeding

The flat-plate BAL device consisted of two plates fabricated of polycarbonate as described previously (Shito et al., 2003). The glass surface comprising the lower plate of the BAL was coated with 0.2 mg/mL rat tail collagen and incubated at 37° C. for 1 h. ESC-derived hepatocyte-like cells (d25-d28) or fibroblasts (3T3-J2) were seeded onto the glass surface at an average density of 20 million cells per seeding. A second seeding of the hepatocyte-like cells was performed after a 1-h incubation period. The cultures were maintained in hepatocyte culture medium supplemented with oncostatin-M, dexamethasone, and insulin/transferrin/selenious acid as noted above for two days. On day three after seeding, the medium was aspirated from the lower plate and the BAL was assembled. The BAL device and perfusion circuit were primed with 6 mL of sterile, heparinized Sprague-Dawley rat plasma (Rockland, Gilbertsville, Pa., USA).

### Example 7

### Extracorporeal Perfusion System

Arterial blood was pumped at 0.55-0.85 mL/min through #13 Masterflex silicone tubing (Cole-Parmer, Vernon Hills, Ill., USA) using a digital peristaltic pump (Cole-Parmer). A plasma separator (mixed cellulose esters MicroKros, 0.2 μm pore size, 16 cm² surface area; Spectrum Labs, Laguna Hills, Calif., USA) was placed after the pump as an interface between the animal blood line and the BAL device line. Separated plasma was pumped through the BAL by means of two peristaltic pumps set at a flow rate of 0.1 mL/min. Separated plasma and blood were reunited before entering a bubble trap, and the reconstituted blood returned to the animal through the venous cannula. During perfusion, heparin (41.5 U/mL) with 5% dextrose solution was administered continuously through the venous line at a rate of 0.2 mL/h via a syringe infusion pump. The dead volume of the entire perfusion system was 12 mL, of which 6 mL was accounted for by the BAL device. Oxygenated gas (21% O₂, 5% CO₂, 74% N₂) flow was established through a chamber above the internal gas permeable membrane of the BAL device.

### Example 8

### Biochemical Analysis of Liver Damage

Blood metabolites were measured using the Piccolo-Portable Blood Analyzer (Abaxis, Union City, Calif., USA). A blood sample of 100 μl volume was collected from the BAL perfusate following the 10 h extracorporeal treatment and used to measure liver enzymes in the Piccolo cartridge.

### Example 9

### Immunofluorescence Analysis

For immunostaining, cultures were washed twice with phosphate-buffered saline (PBS), fixed in 4% paraformaldehyde in PBS at room temperature for 30 min. The fixed sample was then washed twice in PBS, permeabilized using 0.2% Triton X-100 in PBS for 10 min at room temperature. The permeabilized cells were then incubated in blocking buffer (PBS/3% BSA/5% donkey serum) for 60 min at room temperature to block nonspecific antibody binding. Following incubation, the cells were stained for 2 h at room temperature with the following primary antibodies: rabbit anti-Foxa2 (R&D Systems, Minneapolis, Minn., USA), goat anti-AFP (Santa Cruz Biotechnology, Santa Cruz, Calif., USA), rabbit or goat antialbumin (ICN Pharmaceuticals, Aurora, Ohio., USA), mouse anti-CK-18 (Sigma), or isotype-matched antibodies as controls (Santa Cruz Biotechnology). After washing twice in blocking solution, the cells were incubated with the following secondary antibodies: FITC or Cy3-conjugated rabbit IgG, goat IgG, or mouse IgG (ICN Pharmaceuticals), for 60 min at room temperature. For bromodeoxyuridine (BrdU) staining, the cells were incubated with 10 μM BrdU (Sigma) in the culture medium for 24 h at 37° C. and fixed in 70% ethanol for 45 min at room temperature, then treated with 4N HCl for 20 min at room temperature to denature DNA. After incubation in blocking buffer for 30 min, the cells were stained for 60 min at 37° C. with anti-BrdU-Alex594 (Invitrogen, Carlsbad, Calif., USA). In some cases, cells were counterstained with 4,6-diamidino-2-phenylindole (DAPI; Invitrogen) for nuclear staining. Cells were visualized by fluorescence microscopy (Zeiss, Thornwood, N.Y., USA).

Alternatively, cell culture samples were fixed in 4% paraformaldehyde in PBS for 20 minutes at room temperature (RT). The samples were washed twice with PBS and permeabilized using 0.4% Triton X-100 for 10 minutes at RT. Non-specific interactions were blocked using 3% BSA/5% donkey serum in PBS for 1 hour at RT. Cells were incubated for 2 hours at RT or overnight at 4° C. with the following primary antibodies: rabbit anti-albumin (ICN Pharmaceuticals, Aurora, Ohio), goat anti-AFP (Santa Cruz Biotechnology), and mouse anti-cytokeratin 18 (Chemicon). After washing twice with PBS, the cell samples were incubated for 1 hour with the following secondary antibodies: FITC or Texas Red-conjugated anti-rabbit, anti-goat, and anti-mouse IgG's (Cappel Laboratories, Malvern, Pa.). The labeled cell cultures were washed twice and mounting medium with DAPI (Vector Laboratories, Burlingame, Calif.) was placed on the cell cultures to prevent photobleaching.

### Example 10

### FACS Analysis

Cocultured ES-D3 cells were isolated with dispase and trypsin treatment on day ten. Collected cell suspensions were fixed, permeabilized, and stained with antibodies as described above. The primary and secondary antibody concentrations used were the following: rabbit anti-Foxa2, 1:1000 (R&D Systems), goat anti-AFP, 1:1000 (Santa Cruz), donkey antirabbit IgG-FITC, 1:1000 (ICN Pharmaceuticals), and donkey anti-goat IgG-FITC, 1:1000 (ICN Pharmaceuticals). Cell suspensions were analyzed by flow cytometry (Becton Dickinson). For each analysis, 10,000 events were recorded. FACScan data were analyzed using CellQuest software (Becton Dickinson). For flow cytometric analysis of Oct4-GFP ES-R1 cells, cultured ES cells were isolated with dispase and trypsin treatment on day 5 of differentiation. Collected cell suspensions were analyzed by flow cytometry for Oct4-GFP expression. Undifferentiated Oct4-GFP ES-R1 cells were used as a positive control.

Alternatively, Cells were detached from the collagen surface using 10 U/ml collagenase and 1 U/ml dispase in serum-free DMEM for 30 minutes. Cells were then fixed in 4% paraformaldehyde and stored at 4° C. for later analysis. Collected cells were analyzed by flow cytometry (Epics Altra; Becton Dickinson). FACS data was analyzed using CellQuest software. Day-ten ES cells were used as negative controls.

### Example 11

### Functional Analysis

The culture medium samples were collected and stored −20° C. for analysis for urea content. Urea content was determined with diacetylmonoxime with a commercially available kit (StanBio Laboratory, Boerne, Tex., USA). The absorbance was measured with a Thermomax microplate reader (Molecular Devices, Sunnyvale, Calif., USA).

### Example 12

### Quantitative Image Analysis

To quantify the growth of differentiating ES cells, the surface area of cultured ES cells was measured and quantified by Sigmascan Pro image software. Measured surface areas were normalized to the control condition. Three to four random fields of image per sample were acquired and quantified by image analysis. Two independent experiments in duplicate were performed, and the data were represented as an average with the sd.

### Example 13

### RNA Isolation and RT-PCR Analysis

RNA isolation was performed using the Nucleospin RNAII protocol (Clontech, Mountain View, Calif., USA). Following the removal of genomic DNA with DNase, the column was washed and RNA was eluted with distilled water. The RNA purity was quantified using the absorbance ratio at 260 nm (nucleic acids) and 280 nm (protein) and was greater than 1.9. One-Step RT-PCR kit (Qiagen, Valencia, Calif., USA) was used to analyze gene expression. Reactions were initiated using 10 ng RNA and 0.6 μM primer. RT reaction was conducted at 50° C. and 95° C. for 15 min. Three-step cycling was performed: denaturation at 94° C. for 30 s, annealing at 55° C. for 30 s, and extension at 72° C. for 1 min, with a final extension time of 10 min at 72° C. The number of cycles varied between 30 and 35. Following PCR, samples were run on a 2% agarose gel, stained with ethidium bromide, and imaged using the Fluor-X Multiimager (Bio-Rad, Hercules, Calif., USA). Mouse liver total RNA (Ambion, Austin, Tex., USA) was used as a control for hepatocyte gene expression. Markers: Foxa2, AFP, Sox 17, Albumin, GATA1, Runx2, Foxf1, NKx2.5, Tal1, Pax 6, AAT, CK8, CK18, TTR, CYP3A13, Brachyury, Goosecoid, β-actin.

### Example 14

### Statistical Analysis

Data are expressed as the mean±sd. Statistical significance of quantitative image analysis was determined by a two-tailed Student's t test (P<0.001). Animal survival data were evaluated using the generalized Wilcoxon's test and a P value of less than 0.05 was considered statistically significant. Statistical differences of the biochemical analysis were determined by a two-tailed Student's t test (P<0.05).

### Example 15

### Glycogen Staining

Periodic acid-Schiff staining kit was purchased from Sigma. Cells were fixed with 4% paraformaldehyde in PBS for 20 minutes at room temperature. Staining procedures were performed according to manufacturer's instruction.

### Example 16

### Albumin and Urea Assays

Collected medium samples were analyzed for secreted concentrations of mouse albumin by ELISA. Peroxidase-conjugated and affinity-purified anti-mouse albumin and reference mouse albumin were from Bethyl Laboratories (Montgomery, Tex.). Urea content was determined with diacetyl monoxime using a commercially available kit (StanBio Laboratory, Boerne, Tex.). The absorbance was measured with a Thermomax microplate reader (Molecular Devices, Sunnyvale, Calif.).

### Example 17

### EROD Assay

The drug metabolizing activity of the ES cell-derived hepatocytes were evaluated using an ethoxyresorufin 0-de-ethylase (EROD) assay. Day thirty-four ES cells were stimulated with 2 mM 3 methycholanthrene (3-MC) for two days. On day 36, the media was replaced with 10 μM ethoxyresorufin (Invitrogen) and 80 μM dicumarol (Sigma) in Earle's Balanced Salt Solution. Solution samples were collected at 5, 15, 25, and 35 minutes after the introduction of substrates. The increase in fluorescence intensity caused by conversion of ethyoxyresorufin to fluorescent resorufin estimates the CYP1A1 activity in hepatocytes.

1. A method to differentiate pluripotent stem (PS) cells into mature functional hepatocytes, comprising:
(a) differentiating PS cells into monolayer colonies of early endodermal cell populations, and
(b) maturating of the endodermal cell population into hepatic lineage cells expressing hepatocyte lineage markers and/or demonstrating hepatocyte function.

2. The method of claim 1, wherein the maturation of the endodermal cell population is initiated by contacting endodermal progenitor cells with hepatic maturation medium containing dimethyl sulfoxide (DMSO), retinoic acid (RA), and/or nicotinamide

3. The method of claim 2, wherein DMSO is present at a concentration between 1% and 10%.

4. The method of claim 2, wherein RA is present between 0.5 μM and 10 μM.

5. The method of claim 2, wherein nicotinamide is present between 5 mM and 20 mM.

6. The method of claim 2, wherein the hepatic maturation media contains glucose at a concentration between 0.5 g/L and 1.5 g/L.

7. The method of claim 6, wherein the hepatic maturation media contains dexamethasone at a concentration between 50 nM and 500 nM.

8. The method of claim 1, wherein the PS cell-derived hepatocytes express cytokeratin 18, albumin, α1-antitrypsin, glucose-6-phosphatase, glycogen storage capabilities, and/or cytochrome P450 activity.

9. The method of claim 1, wherein the PS cell-derived hepatocytes demonstrate similar metabolic activites as primary hepatocytes selected from the group consisting of gluconeogenesis, glycogenolysis, ureagenesis, and ketogenesis.

10. The method of claim 1, wherein the PS cells are embryonic stem cells or somatic cells reprogrammed into a pluripotent state.

11. The method of claim 10, wherein the PS cells are human PS cells.

12. The method of claim 10, wherein the PS cells are mouse PS cells.

13. The method of claim 1, wherein the PS cell-derived hepatocytes secrete plasma proteins selected from the group consisting of albumin, α1-antitrypsin, transferrin, blood coagulation factor VII, factor VIII, factor XII, and protein C.

14. A method for treatment of patients for injuries, diseases, or conditions associated with impaired liver function comprising implantation of a differentiated hepatocytes produced by the method of claim 1.
